(19) United States
(12) Patent Application Publication
(10) Pub. No.: US 2026/0250336 A1
(43) Pub. Date: Aug. 27, 2026

(54) ANTI-CANCER STRAIN SECRETING STREPTAVIDIN-FUSION PROTEIN HAVING BIOTIN BINDING ABILITY, ANTI-CANCER COMPOSITION USING THE SAME, ANTI-CANCER ADJUVANT AND TUMOR IMAGING AGENT

(71) Applicants: THE INDUSTRY & ACADEMIC COOPERATION IN CHUNGNAM NATIONAL UNIVERSITY (IAC), Daejeon (KR); KOREA INSTITUTE OF SCIENCE AND TECHNOLOGY, Seoul (KR)

(72) Inventors: Hyo Jin LEE, Daejeon (KR); Heung Jin JEON, Daejeon (KR); Seung Beum SUH, Seoul (KR); Sehoon KIM, Seoul (KR); Dohee LEE, Seoul (KR)

(73) Assignees: THE INDUSTRY & ACADEMIC COOPERATION IN CHUNGNAM NATIONAL UNIVERSITY (IAC), Daejeon (KR); KOREA INSTITUTE OF SCIENCE AND TECHNOLOGY, Seoul (KR)

(21) Appl. No.: 18/993,927

(22) PCT Filed: Jun. 29, 2023
(86) PCT No.: PCT/KR2023/009113

§ 371 Date: Jan. 13, 2025

(30) Foreign Application Priority Data
Jul. 20, 2022 (KR) 10-2022-0089805

(51) Int. Cl. C07K 14/36; A61K 35/74; A61K 49/18; C07K 14/195; C12N 1/20; C12N 15/74; C12R 1/42

(52) U.S. Cl. CPC C07K 14/36; A61K 35/74; A61K 49/1896; C07K 14/195; C12N 1/20; C12N 15/74; C07K 2319/43; C12R 2001/42

(57) ABSTRACT

The present invention relates to a gene construct characterized by encoding a fusion protein of flgM and Strepavidin having biotin binding ability through the linkage of an anti-sigma factor flgM gene and a gene encoding streptavidin, which enables expression and secretion of a streptavidin-fusion protein with biotin binding ability in an anti-cancer strain capable of targeting tumor sites and stimulating immune activity, and to an anti-cancer adjuvant and tumor imaging agent that, when transformed by this construct, exhibits anti-cancer activity itself and can be effectively utilized in the diagnosis and treatment of tumor cells together with anti-cancer substances or contrast agents labeled with biotin, which specifically interacts with streptavidin.

### CROSS-REFERENCE TO PRIOR APPLICATIONS

This Application is a National Stage Patent Application of PCT International Application No. PCT/KR2023/009113 (filed on Jun. 29, 2023), which claims priority to Korean Patent application No. 10-2022-0089805 (filed on Jul. 20, 2022), which are all hereby incorporated by reference in their entirety.

### SEQUENCE LISTING

This application contains a Sequence Listing submitted via XML file and hereby incorporated by reference in its entirety. The sequence listing is named “782-0017_Sequence_Listing.xml”, created on Jan. 9, 2025 and 8,890 bytes in size.

### BACKGROUND

The present invention relates to a gene construct that enables expression and secretion of a streptavidin-fusion protein with biotin binding ability in an anti-cancer strain capable of targeting tumor sites and stimulating immune activity, and to an anti-cancer adjuvant and tumor imaging agent that, when transformed by this construct, exhibits anti-cancer activity itself and can be effectively utilized in the diagnosis and treatment of tumor cells together with anti-cancer substances or contrast agents labeled with biotin, which specifically interacts with streptavidin.

Cancer is a disease that threatens life by destroying normal cells as cell proliferation activity does not stop and invades surrounding tissues. As the body's regulatory ability decreases due to aging, it becomes vulnerable to cancer, and in modern aging society, cancer has ranked first in overall causes of death since 2007, exceeding twice the mortality rate of heart disease, which ranks second.

While strengthening immunity is the most effective response system for treating diseases, cancer cells evade the body's immune response because they are not external invaders. Certain viruses or bacteria infect cancer cells more than normal cells, and infected bacteria can become targets of immune cells. Accordingly, anti-cancer treatment methods have been proposed that stimulate the body's immune response to combat cancer cells by deliberately infecting with certain viruses or bacteria. However, infectious bacteria such as Shigella, Vibrio cholerae, and pathogenic E. coli only infiltrate intestinal cells and do not reach important organs that cause immune responses such as the liver and spleen. In contrast, Salmonella can stimulate systemic immune responses by infiltrating the spleen and liver through lymph nodes. Specifically, Leschner et al. (J. Mol. Med. 2010, 88, 763-773) traced the infection route in mice with CT26 tumors over time after intravenous injection of fluorescent Salmonella. The results showed that immediately after infection, the entire body was infected through the mouse's blood, Salmonella accumulated in the spleen and liver 20 minutes after infection, and after 24 hours, Salmonella was observed to be concentrated only in tumor tissue. This indicates that Salmonella may be more effective in anti-cancer immunotherapy compared to other strains, as it stimulates systemic immune responses in the spleen and liver and subsequently targets cancer to activate localized immunity.

However, Salmonella is a representative bacterium that causes food poisoning, and if infected in tissues other than tumors, it can threaten life by causing sepsis due to abnormal host responses. A research team at Yale University in the United States reported that genetic manipulation of Salmonella can attenuate toxicity while maintaining tumor-attacking properties, and that injection of the attenuated Salmonella can suppress tumors by inducing immune stimulation. Accordingly, extensive research is underway to develop attenuated Salmonella strains engineered to express substances with anti-cancer properties. However, attenuated Salmonella exhibits low survival and reduced immunogenicity, and there is a risk that mutations could revert attenuated strains to wild-type Salmonella, leading to sepsis, necessitating additional safety measures.

To complement the low immunogenicity of attenuated Salmonella, studies have been conducted to develop Salmonella strains that can additionally express or suppress substances that help anti-cancer treatment, and to use them for anti-cancer treatment. Korean Patent No. 10-0852687 discloses a Salmonella strain expressing tumor necrosis factor alpha through the introduction of a tumor necrosis factor alpha protein vector into an attenuated Salmonella, along with an anti-cancer therapeutic composition containing this strain, and Korean Patent No. 10-1750007 discloses a solid cancer therapeutic agent based on an attenuated Salmonella strain engineered to selectively express L-asparaginase at tumor sites. Although the above Salmonella strains additionally complemented anti-cancer activity by expressing anti-cancer substances, they still carry the risk of causing sepsis by converting attenuated strains to wild-type Salmonella. In addition, when expressing anti-cancer substances in vivo, the Salmonella strain itself may be killed by the physiological activity of the anti-cancer substances, so for simultaneous expression of anti-cancer substances and Salmonella's anti-cancer efficacy, the Salmonella strain should be able to stably survive after targeting the tumor site and continuously express and secrete anti-cancer substances.

Streptavidin has very fast binding rate with biotin and also very strong binding affinity (K_{d}=10⁻¹⁵ M), and because it is less affected by pH, temperature, or organic solvents, it is useful for detection or purification of specific proteins. Korean Patent Application Publication No. 10-2022-0058461 disclosed that cancer diagnosis can be performed by introducing a gene encoding streptavidin into a host cell targeting cancer. However, the above patent noted that introducing only the streptavidin gene resulted in low expression levels, and that adding maltose binding protein to enhance expression weakened its binding affinity with biotin, so streptavidin expression could only be increased while maintaining biotin binding ability through a specific gene construct structure. Furthermore, according to that gene construct, streptavidin remained in the host cell's periplasm and was not secreted. However, when streptavidin remains confined within the strain, it binds only to biotin-containing substances absorbed into the strain, thereby reducing its usability, and especially when used as an anti-cancer adjuvant, externally administered anti-cancer substances are targeted to the inside of host cells rather than tumor cells, which makes it difficult to achieve anti-cancer efficacy. Also, as expressed streptavidin cannot be secreted and accumulates in host cells, it can affect host cell survival.

### SUMMARY

The present invention aims to solve the problems of the prior art by providing a gene construct that enables stable expression and secretion of a fusion protein having biotin binding ability.

Another object of the present invention is to provide an anti-cancer recombinant strain, which is transformed by the above construct to secrete a fusion protein with biotin binding ability, and is targeted to tumor sites while exhibiting immune anti-cancer activity against tumors itself, and an anti-cancer composition comprising the same.

A further object of the present invention is to provide an anti-cancer adjuvant or tumor imaging adjuvant that can enhance anti-cancer efficacy or imaging efficacy by targeting anti-cancer substances bound with biotin administered externally or contrast agents bound with biotin for tumor imaging to tumor sites.

The present invention also aims to provide a method for providing information for cancer diagnosis by utilizing the above tumor imaging adjuvant.

To achieve the aforementioned objectives, the present invention relates to a gene construct characterized by encoding a fusion protein of flgM and streptavidin (hereinafter abbreviated as “Stv”) through the linkage of an anti-sigma factor flgM gene and a gene encoding streptavidin. In particular, the flgM-Stv expressed by the gene construct of the present invention is characterized by its binding ability to biotin.

Unless otherwise defined, all scientific and technical terms used in this specification are defined to have the same meanings as commonly understood by those skilled in the art to which this invention belongs. Furthermore, in explaining the invention, detailed descriptions of known technologies related to the invention will be omitted if it is determined that they may unnecessarily obscure the gist of the invention.

The flgM-Stv expressed by the gene construct of the present invention was able to bind biotin similarly to streptavidin, either in its secreted form or within the strain expressing flgM-Stv, thereby demonstrating the biological equivalence of streptavidin and flgM-Stv in terms of biotin binding ability.

In the gene construct of the present invention, the flgM gene and gene encoding Stv may be directly linked or linked via a linker. The examples below exemplified constructs of SEQ ID NO: 1 or 2 connected via a linker having a gene sequence encoding an amino acid sequence consisting of HIS*6-SGG (SEQ ID NO: 5) or GGSSSS-HIS*6-SGG (SEQ ID NO: 6), but these are not limiting. The examples below demonstrated that flgM-Stv was expressed in both strains regardless of linker length, and expression efficiency could vary depending on linker length. Therefore, it is natural that additional linker optimization could be performed to show superior expression efficiency, but those skilled in the art would readily modify the length or sequence of the linker appropriately.

To enhance expression efficiency, a Shine-Dalgarno sequence may be positioned upstream of the gene construct of the present invention. It is also evident that appropriate restriction enzyme sequences can be positioned at the upstream and downstream regions to facilitate the introduction of the gene construct into a vector. The examples below include a his-tag in the middle and a flag-tag at the terminus to facilitate detection of the fusion protein, but this is not essential, and the type of tag can be modified as needed.

The present invention further relates to a recombinant expression vector comprising the above gene construct. In the present invention, a “vector” refers to a DNA construct operably linked to enable the expression of the gene construct in a suitable host, which may be a plasmid, phage particle, or potential genomic insert. In the specification of the present invention, the terms plasmids and vectors are sometimes used interchangeably. It is natural that the expression vector can be selected and used with high efficiency according to the sequence of the gene construct.

In addition, the present invention relates to an anti-cancer recombinant strain that is transformed with the above gene construct operably linked to an inducible promoter, wherein the strain is characterized by expressing and secreting flgM-Stv with biotin binding ability and targeting tumor cells. The strain of the present invention is known to exhibit immune anti-cancer activity by targeting tumors, and any strain that is harmless to humans can be used. Examples of such strains include E. coli, attenuated Salmonella, Shigella, Acinetobacter, Pseudomonas, Listeria, Clostridium or intestinal microorganisms (beneficial bacteria), probiotics, etc., but are not limited thereto.

The recombinant strain of the present invention can be transformed with a recombinant expression vector comprising the above gene construct. The transformation method follows conventional techniques and can use, for example, calcium chloride method, electroporation, etc., but is not limited thereto. The recombinant strain of the present invention is characterized by not only expressing flgM-Stv encoded by the gene construct of the present invention simply inside cells by the operation of flgM, but also secreting it outside the strain. As flgM-Stv is secreted, even if the strain continuously expresses flgM-Stv, it does not accumulate beyond a certain concentration in vivo, so strain survival is not threatened by expressed flgM-Stv, enabling stable expression.

In addition, the strain of the present invention can more extensively utilize the binding between flgM-Stv and biotin by secreting flgM-Stv with biotin binding ability. If flgM-Stv is expressed and remains only inside the strain, substances bound with biotin administered externally must enter the strain to bind with biotin. Therefore, substances bound with biotin accumulate inside the strain and do not come into directly contact tumor cells. In contrast, if the strain not only expresses flgM-Stv when targeted to tumors but also secretes it to tumor cells, biotin-bound substances can be targeted to tumors to directly act on tumor cells. Even if some secreted flgM-Stv diffuses, it will still exist at highest concentration in tumors where the strain is targeted. Moreover, since avidin-like proteins are known to be rapidly degraded in serum when injected in vivo, flgM-Stv secreted by the strain and diffused to areas outside tumors is also quickly degraded and loses biotin binding ability, so it does not affect targeting of biotin-bound substances.

In this specification, “operably linked to an inducible promoter” refers to the ability to switch the operation of the linked gene using an inducing substance. The inducible promoter may be, for example, tac promoter, lac promoter, lacUV5 promoter, lpp promoter, pLλ promoter, pRλ promoter, rac5 promoter, amp promoter, recA promoter, SP6 promoter, trp promoter, T7 promoter, pBAD promoter, Tet promoter, trc promoter, pepT promoter, sulA promoter, pol II (dinA) promoter, ruv promoter, uvrA promoter, uvrB promoter, uvrD promoter, umuDC promoter, lexA promoter, cea promoter, caa promoter, recN promoter, pagC promoter, hip promoter, ansB promoter, or pflE promoter.

In particular, the strain of the present invention may be an attenuated Salmonella strain. Unlike other strains, Salmonella strains can infiltrate the liver and spleen to stimulate systemic immune responses, so their immune anti-cancer activity is even more excellent. “Attenuation” refers to the artificial weakening of a living pathogen's toxicity by mutating genes involved in essential metabolism so that it cannot cause disease in the body and only stimulates the immune system to induce immunity. Salmonella attenuation-inducing genes are well known in the art and can be achieved by loss of function of one or more genes selected from the group consisting of aroA, aroC, aroD, aroE, Rour, htrA, ompR, ompF, ompC, galE, cya, crp, cyp, phoP, phoQ, rfaY, dksA, hupA, sipC, clpB, clpP, clpX, pab, nadA, pncB, pmi, rpsL, hemA, rfc, poxA, galU, cdt, pur, ssa, guaA, guaB, fliD, flgk, flgL, relA and spol. To prevent reversion to wild-type during in vivo application and further weaken toxicity, the functions of two or more of the above genes may be disrupted.

In particular, when flgM-Stv is expressed in attenuated Salmonella strains, it was observed that flagella disappeared resulting in inhibited motility or even complete loss of motility. This indicates that safety is significantly improved because movement to other organs is restricted by expressing flgM-Stv after targeting the tumor site. Specifically, the strain may be S-flgM-streptavidin-pBAD18-ASD+/BRD509 asd with deposit number KCTC15482BP, deposited at the Korean Collection for Type Cultures of the Korea Research Institute of Bioscience and Biotechnology on Jun. 26, 2023, but is not limited thereto.

The present invention further relates to an anti-cancer composition comprising the above anti-cancer recombinant strain as an active ingredient. The anti-cancer recombinant strain of the present invention can be used as an anti-cancer composition because it naturally targets tumor sites itself and exhibits immune anti-cancer activity.

The composition of the present invention can be prepared using established methods in the pharmaceutical field for use as an anti-cancer agent, and can be used alone or mixed with pharmaceutically acceptable carriers, forming agents, diluents, etc.

The composition of the present invention can be formulated into preparations for oral or parenteral administration. The dosage of the active ingredient according to the present invention can be appropriately adjusted according to absorption of active ingredients in vivo, formulation type, age, gender and condition of patients, degree of symptoms, etc., and can be administered once a day or divided into multiple doses. The typical dosage ranges from 0.001 mg/kg·day to 10 g/kg·day.

In addition, the present invention relates to an anti-cancer adjuvant characterized by comprising the above anti-cancer recombinant strain as an active ingredient and being co-administered with an anti-cancer agent labeled with a substance that specifically binds to Stv. Examples of substances that specifically bind to Stv include biotin, but are not limited thereto.

As described above, since the strain of the present invention secretes flgM-Stv having biotin binding ability to tumor cells after targeting tumor cells by inducing agent, when co-administered with anti-cancer agents in biotin-bound form, the above anti-cancer agents can be targeted to tumor cells. Therefore, side effects of anti-cancer agents on other organs can be minimized and anti-cancer activity can be enhanced, and anti-cancer activity can be shown even at lower doses, reducing the burden on patients caused by side effects. In this way, the recombinant strain of the present invention can be used as an anti-cancer adjuvant co-administered with anti-cancer agents in biotin-bound form. As the anti-cancer agent, any substance that has little or no ability to target tumor cells itself while exhibiting anti-cancer activity can be used as long as it can be labeled with substances that specifically bind to Stv, such as nanoparticles, antibodies, anti-cancer proteins or small molecule substances. As demonstrated in the tumor imaging results using dyes in the examples below, the recombinant strain of the present invention can inhibit the excretion of anti-cancer agents through binding between flgM-Stv and biotin, enabling anti-cancer agents to act as sustained-release formulations.

The anti-cancer recombinant strain of the present invention can also be utilized as a tumor imaging adjuvant that can enhance tumor tissue imaging efficacy by co-administration with contrast agents in biotin-bound form, thereby targeting the contrast agent to tumor sites. As the contrast agent, one or more selected from the group consisting of radioactive nuclides, fluorescent labels, enzyme labels, chemiluminescent markers, gold preparations and magnetic preparations can be used, and determining the imaging method based on the type of contrast agent would be easy for those skilled in the art. The tumor imaging adjuvant of the present invention not only enhances tumor imaging efficiency but also exhibits anti-cancer activity itself, so it can have more beneficial effects for cancer patients.

The tumor imaging adjuvant of the present invention can be effectively used for providing information for cancer diagnosis. For example, providing information for cancer diagnosis can be carried out by a method comprising: (A) targeting tumor cells by administering the tumor imaging adjuvant of the present invention to a patient suspected of having cancer or undergoing anti-cancer treatment; (B) inducing expression of flgM and streptavidin fusion protein in the recombinant strain of the tumor imaging adjuvant; (C) administering a contrast agent in biotin-bound form; and (D) detecting and imaging the contrast agent. However, it is not limited to the above steps, and appropriate modifications can naturally be made based on the type and characteristics of the strain and contrast agent, for instance, steps (B) and (C) may be carried out simultaneously, or steps (A) and (C) may be carried out simultaneously, or additional steps may be added.

As described above, the gene construct of the present invention, when introduced into an anti-cancer strain that targets tumor cells and shows immune anti-cancer activity, allows the strain to secrete flgM-Stv with biotin binding ability.

Therefore, the anti-cancer recombinant strain introduced with the gene construct of the present invention can not only exhibit anti-cancer activity itself, but also can be used as an anti-cancer adjuvant or tumor imaging adjuvant that targets anti-cancer agents or contrast agents to tumors through specific binding between biotin and streptavidin when co-administered with anti-cancer agents or contrast agents that have no tumor targeting function but are in biotin-bound form, thereby enhancing anti-cancer efficacy and tumor imaging efficacy.

### BRIEF DESCRIPTION OF THE DRAWINGS

FIG. 1 is a diagram showing amino acid sequences of monomeric streptavidin and linker.

FIG. 2 is a diagram showing DNA base sequences designed for preparing plasmids for expression of flgM-Stv fusion protein in one embodiment.

FIG. 3 is a diagram showing DNA base sequences designed for preparing plasmids for expression of flgM-Stv fusion protein in another embodiment.

FIG. 4 is an electrophoresis photograph quantifying flgM-Stv in the culture medium of strains transformed with the above plasmids.

FIG. 5 is a schematic diagram and a graph showing the results of HABA assay that demonstrates biotin binding ability of flgM-Stv secreted by a strain in one embodiment of the present invention.

FIG. 6 is fluorescence microscope images and quantification graphs showing biotin uptake assay results for a strain in one embodiment of the present invention.

FIG. 7 is images and graphs showing tumor imaging results using a strain in one embodiment of the present invention.

FIG. 8 is images showing inhibition of strain motility by induction of flgM-Stv expression.

FIG. 9 is TEM images showing morphology changes of strain according to induction of flgM-Stv expression.

### DETAILED DESCRIPTION

The present invention is described in more detail below with reference to the accompanying drawings and examples. However, these drawings and examples are only illustrative to easily explain the content and scope of the technical idea of the present invention, and the technical scope of the present invention is not limited or altered thereby. Based on such examples, various modifications and changes are possible within the scope of the technical idea of the present invention, which would be obvious to those skilled in the art.

### EXAMPLES

### Example 1: Construction of Plasmids for Expression of Streptavidin Fusion Protein

Plasmids were constructed by fusing flgM protein, which is secreted extracellularly through the basal body and hook, with Stv.

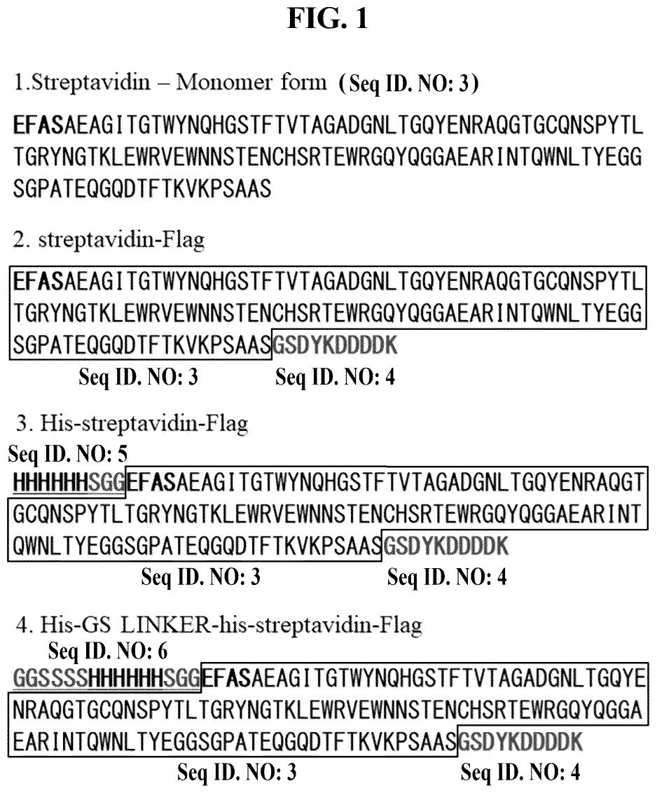

First, genes capable of expressing fusion proteins of flgM and Stv were designed. Flag tag sequence (SEQ ID NO: 4) was added to monomeric Stv amino acid sequence (SEQ ID NO: 3) for detection, and linkers containing 6 histidines (SEQ ID NO: 5) or GS linker containing 6 histidines (SEQ ID NO: 6) were used as linkers for fusion with flgM protein. FIG. 1 illustrates the linkage between the linker, monomeric Stv, and flag-tag.

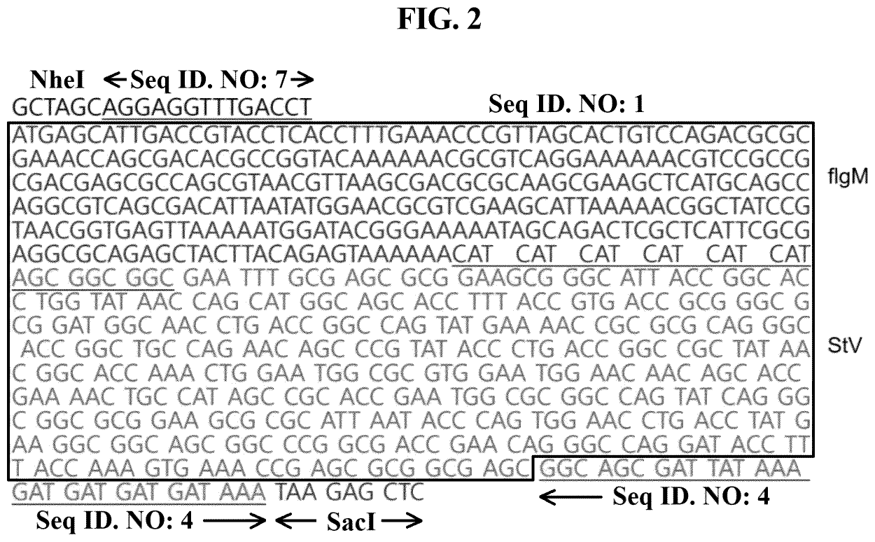

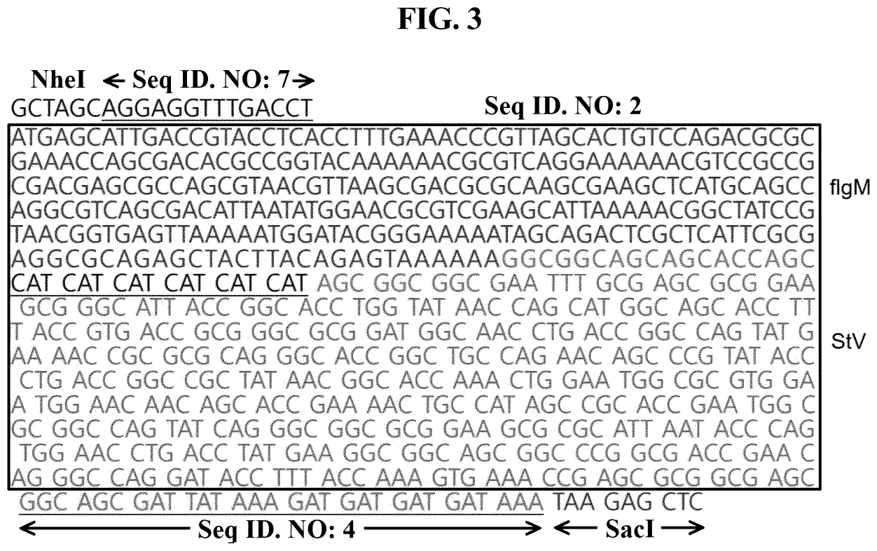

The DNA sequence of wild-type Salmonella flgM (flgM anti-sigma-28 factor FlgM [Salmonella enterica subsp. enterica serovar Typhimurium str. LT2] Gene ID: 1252690, Locus tagSTM1172 NC_003197.2 (1257036 . . . 1257341, complement)) and the DNA sequence obtained by Salmonella codon-optimizing the amino acid sequence of monomeric streptavidin (His-streptavidin-Flag or His-GS LINKER-streptavidin-Flag in FIG. 1) were combined. To increase expression of the flgM gene, 14 Shine-Dalgarno sequences (SEQ ID NO: 7) were inserted in front of the flgM gene, and for cloning, NheI site was inserted at the very front, and SacI site was inserted after the stop codon of flag tag. FIGS. 2 and 3 show the base sequence of flgM-his-streptavidin-flag tag and flgM-GS LINKER-his-streptavidin-flag tag designed through the above process. The sequences encoding flgM and sequences encoding Stv are shown separately in FIGS. 2 and 3. The gene sequences were synthesized by Cosmogenetech Co., Ltd. The sequences excluding the restriction enzyme sites and Shine-Dalgarno sequences correspond to genes encoding the fusion protein of flgM and Stv (hereinafter referred to as “flgM-Stv”) (SEQ ID NO: 1 and SEQ ID NO: 2).

The synthesized flgM-his-streptavidin-flag tag and flgM-GS linker-his-streptavidin-flag tag were digested with 100 each of NheI (Takara) and SacI (Takara) at 37° C. for 2 hours. The reaction products were electrophoresed and about 760 bp DNA was extracted using a Gel extraction kit (Qiagen) to isolate insert DNA for each fusion protein flgM-Stv.

pBad18 asd plasmid (obtained from Prof. Heon-Man Lim's lab at Chungnam National University) was digested with 10 U each of NheI and SacI enzymes at 37° C. for 2 hours, and the reaction product was purified using a PCR purification kit (Qiagen) to obtain vector DNA. The vector DNA was ligated (Invitron) with the two flgM-Stv inserts at 25° C. for 30 minutes each and transformed into DH5a competent cells. The transformed cells were plated on LB amp solid medium and cultured at 37° C. to select colonies with antibiotic resistance. 6 candidates were selected from each selected colony and digested with 10U each of NheI and SacI at 37° C. for 1 hour, and the formation of 760 bp bands was confirmed by electrophoresis. The nucleotide sequences of the candidates were analyzed by Cosmogenetech Co., Ltd. to confirm the insertion of genes of SEQ ID NO: 1 or 2, and flgM-his-streptavidin-flag tag plasmid and flgM-GS linker-his-streptavidin-flag tag plasmid were successfully constructed for secretion of flgM-Stv.

### Example 2: Preparation of Attenuated Salmonella Strains Secreting flgM-Stv with Biotin Binding Ability

1) Transformation of Attenuated Salmonella Strains and Confirmation of flgM-Stv Secretion

Using each of the flgM-his-streptavidin-flag tag plasmid and flgM-GS linker-his-streptavidin-flag tag plasmid prepared in Example 1, attenuated Salmonella strains were individually transformed, and expression of flgM-Stv fusion protein was confirmed. For the attenuated Salmonella strain, aroA aroD asd strain (obtained from Prof. Heon-Man Lim at Chungnam National University) was used.

A single colony of each transformed strain was inoculated into LB amp liquid medium and shaken cultured at 37° C. for 12-16 hours. Subsequently, it was diluted with fresh LB amp liquid medium to OD₆₀₀ of 0.05 and shaken cultured at 37° C. for 2 hours. When OD₆₀₀ reached 0.4-0.6 by shaking culture, arabinose was treated to a final concentration of 0.2 w %, and shaken cultured at 37° C. for an additional 5 hours. For comparison, the control group was shaken cultured under the same conditions without arabinose treatment. The culture medium was centrifuged at 3000 rpm to separate the supernatant, and filtered through a 0.2 μm filter to completely remove bacteria.

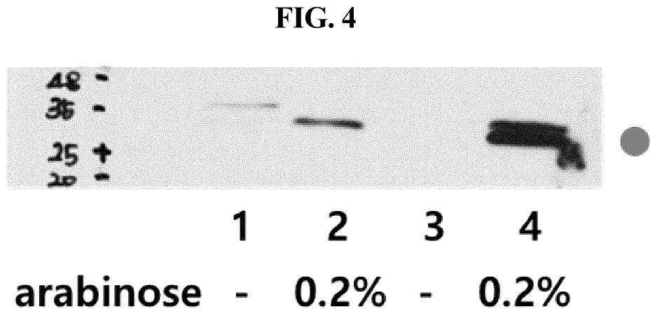

The amount of flgM-Stv in the culture medium was confirmed through Western blot from the bacteria-free supernatant. FIG. 4 is an electrophoresis photograph showing detection results of fusion protein in culture medium of experimental groups with or without arabinose treatment for strains transformed with flgM-his-streptavidin-flag tag plasmid (lanes 1, 2) and flgM-GS linker-his-streptavidin-flag tag plasmid (lanes 3, 4), wherein lanes 1, 3 are from the experimental groups not treated with arabinose and lanes 2, 4 are from the experimental groups treated with arabinose. In FIG. 4, both strains showed detection of fusion proteins in the culture medium when treated with arabinose, and the Salmonella strain transformed by the flgM-GS linker-his-streptavidin-flag tag plasmid showed remarkably superior flgM-Stv fusion protein secretion ability compared to the Salmonella strain transformed by the flgM-his-streptavidin-flag tag plasmid. Accordingly, the strain transformed by the flgM-GS linker-his-streptavidin-flag tag plasmid showing superior flgM-Stv secretion ability was named S-flgM-streptavidin-pBAD18-ASD+/BRD509 asd and deposited at the Korean Collection for Type Cultures of the Korea Research Institute of Bioscience and Biotechnology on Jun. 26, 2023 (deposit number KCTC15482BP).

Below, the efficacy resulting from flgM-Stv fusion protein expression was tested using the S-flgM-streptavidin-pBAD18-ASD+/BRD509 asd strain.

2) Testing Biotin Binding Ability of flgM-Stv (HABA Assay)

Biotin binding ability of flgM-Stv was confirmed using HABA (2-(4′-HydroxyAzoBenzene) Benzoic Acid). Complexes formed by binding between HABA and streptavidin absorb light at 500 nm. Therefore, if flgM-Stv secreted into the culture medium maintains streptavidin's binding ability, it will bind with HABA in solution and increase absorbance at 500 nm. Also, since biotin binds more strongly with streptavidin than HABA, if sufficient biotin exists in solution and flgM-Stv maintains streptavidin's biotin binding ability, flgM-Stv will bind with biotin and not form complexes with HABA, so there will be no observable change in absorbance at 500 nm.

Specifically, a single colony of the attenuated Salmonella strain expressing the flgM-Stv fusion protein prepared in 1) above was put into 20 mL of LB amp liquid medium and cultured at 37° C. for 16 hours. The above culture was added to fresh LB amp liquid medium to achieve an OD₆₀₀ of 0.05, then further cultured for an additional 2 hours at 37° C. until OD₆₀₀ reached 0.4-0.6. Subsequently, 200 μL of 20% (w/v) arabinose was added to achieve a final concentration of 0.2% and culture was incubated at 37° C. while taking 1 mL culture samples at 1-hour intervals. The same amount of PBS was added to the control group. The culture medium was centrifuged at 3000 rpm to separate the supernatant, and the supernatant was filtered through a 0.2 μm filter to completely remove bacteria. 100 μL of HABA reagent and 100 μL of biotin solution (1 mg/50 mL) were added to 100 μL of bacteria-free culture supernatant and mixed at 600 rpm for 1 hour using a vortex. For the control group, 100 μL of PBS was added instead of biotin and mixed under the same conditions. After 1 hour, OD₅₀₀ values were measured.

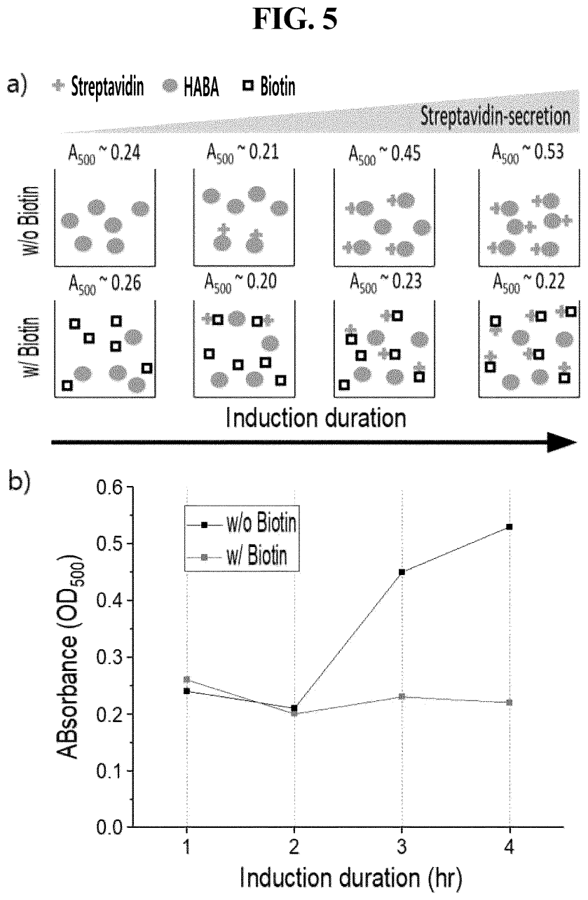

FIG. 5 shows a schematic diagram of biotin binding ability testing of flgM-Stv through HABA assay and its result graph. First, looking at results from samples without added biotin, there was no change in absorbance at 500 nm until 2 hours after arabinose induction, but afterwards absorbance gradually increased showing that HABA had formed complexes. This indicates first, that flgM-Stv has binding ability with HABA like streptavidin, and second, that flgM-Stv begins to be secreted no later than 2 hours after arabinose induction. Meanwhile, when biotin was added to the solution, there was no change in absorbance at 500 nm, proving that flgM-Stv binds strongly with biotin.

From this, it was confirmed that secreted flgM-Stv forms strong binding with biotin like streptavidin.

3) Testing Biotin Binding Ability of flgM-Stv Expressing Strain (Biotin Uptake Assay)

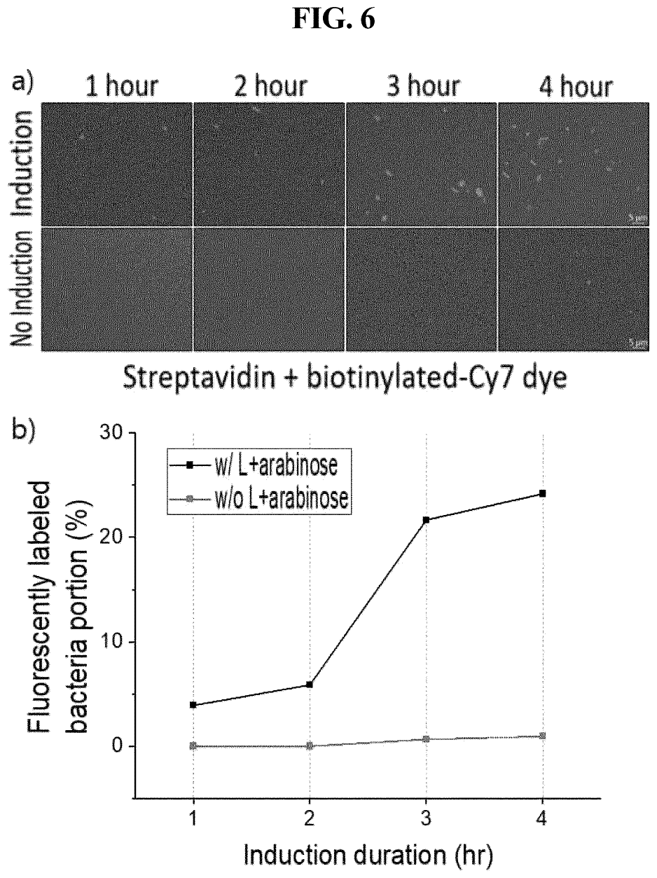

Biotin binding ability of the transformed recombinant strain itself was evaluated through biotin uptake assay. For this, pellets obtained together when centrifuging to obtain supernatant from samples collected at each time point in 2) were used. The strain pellets separated by removing supernatant were resuspended in 1 mL PBS. 100 μL of fluorescent probe (Bioacts cat #RFP0613, excitation/emission wavelength=749 nm/774 nm) solution (1 mg/5 mL) bound with biotin was added and reacted by mixing at 600 rpm for 1 hour using a vortex mixer, then samples were washed and observed with a fluorescence microscope. FIG. 6 shows the fluorescence microscope images and graphs quantified using ImageJ program. From FIG. 6, it was confirmed that strains induced to express flgM-Stv by arabinose showed fluorescence signals due to binding with biotin of fluorescent dye, and the proportion increased according to induction time.

### 4) Tumor Imaging

Testing was performed through in-vivo tumor imaging analysis to confirm that the strain of the present invention can be used for tumor targeting of biotin-bound substances.

Specifically, 4T1 cells (5×10⁵ cells/60 μL PBS) were subcutaneously injected into the left thigh of Balb/c nude mouse (female) and incubated for 2 weeks to induce tumors. S-flgM-streptavidin-pBAD18-ASD+/BRD509 asd-strain was intravenously injected into the mouse in a concentration of 5×10⁶ cfu/60 μL PBS. 72 hours after strain injection, 200 μL of 20% (w/v) arabinose was injected intraperitoneally, and 4 hours later 20 μL of biotin-bound fluorescent probe solution (20 μg/mL), identical to that used in 3), was subcutaneously injected at the tumor site. Subsequently, respiratory anesthesia was performed and fluorescence signals were confirmed by IVIS (in vivo imaging system) at different time points.

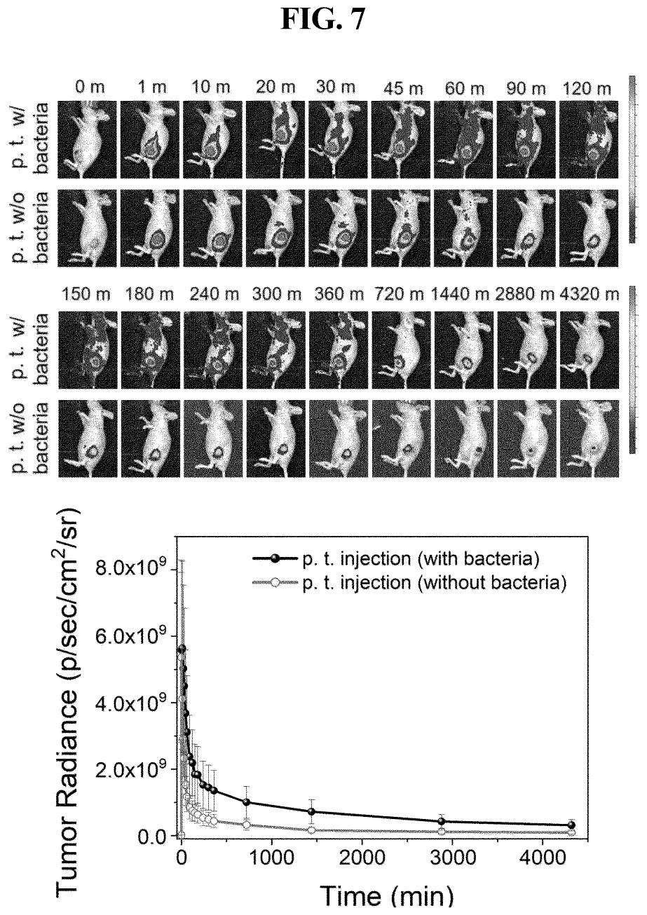

FIG. 7 shows that despite injecting the same amount of fluorescent probe, due to inhibition of dye excretion through binding between biotin and flgM-Stv, not only does the fluorescence signal persist longer, but overall higher signals are observed. This means that even when dramatically reducing the amount of contrast agent needed for tumor imaging, the same imaging sensitivity can be obtained. Also, when used to target anti-cancer agents, it suggests that side effects can be reduced because the same efficacy can be achieved at the tumor site with less anti-cancer agent due to targeting the anti-cancer agent, and it also indicates that sustained-release efficacy can be achieved by inhibiting excretion of the anti-cancer agent.

### 5) Testing Motility of Salmonella Strain

Interesting results were observed during strain culture showing that expression of flgM-Stv affects strain motility. This led to formal testing of strain motility according to flgM-Stv expression using soft agar tryptone plates (per liter: 10 g Bacto tryptone (BD: 211705), 5 g NaCl (Biosesang S1009), and 3 g Bacto agar (BD: 214010)).

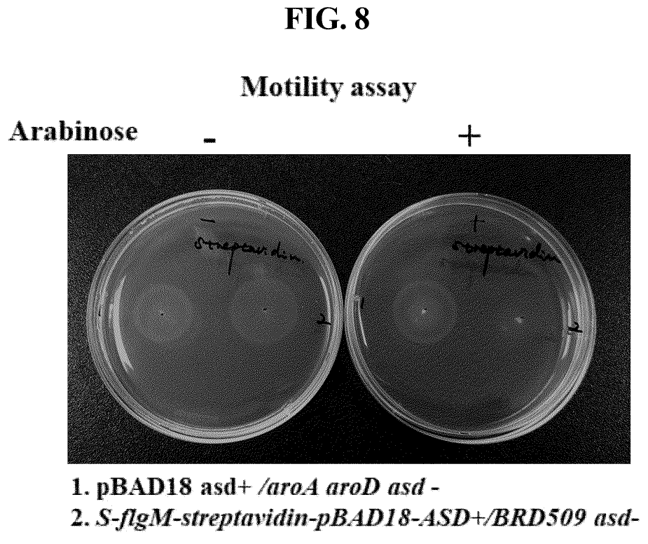

Single colonies of Salmonella strain transformed with pBAD18 asd+/aroA aroD asd-plasmid (1) and S-flgM-streptavidin-pBAD18-ASD+/BRD509 asd-strain (2) were each transplanted at separated positions using sterilized toothpicks on soft agar tryptone plates with or without 0.2% arabinose. Subsequently, motility was evaluated after culturing at 37° C. for 5 hours. FIG. 8 shows the results, confirming that the motility of the Salmonella strain was lost by expression of flgM-Stv.

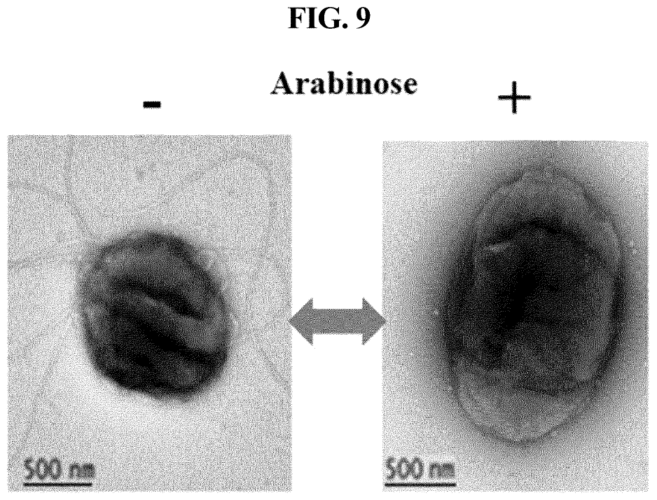

To elucidate the cause of motility loss, strain morphology before and after arabinose induction was observed by TEM. The TEM images in FIG. 9 show that flagella of Salmonella strain disappear as flgM-Stv expression is induced by arabinose. Since Salmonella flagella are organelles responsible for motility, it can be seen that motility also disappears as flagella disappear.

Appendix to the Specification: Receipt in the case of an original deposit

Name of depositor: The Korean Collection for Type Cultures, Deposit date: Jun. 26, 2023, Accession number: KCTC15482BP, the address of the Korean Collection for Type Cultures is Korea Research Institute of Bioscience and Biotechnology (KRIBB) 181, Ipsin-gil, Jeongeup-si, Jeolllabuk-do, 56212, Republic of Korea. The deposit was made under Budapest Treaty, and that all restrictions imposed by the depository will be irrevocably removed upon the granting of the patent. The deposit is hereby incorporated by reference in its entirety.

1. A gene construct characterized by encoding a fusion protein of flgM and Strepavidin having biotin binding ability through the linkage of an anti-sigma factor flgM gene and a gene encoding streptavidin.

2. The gene construct according to claim 1,
wherein the flgM gene and gene encoding streptavidin are directly linked or linked via a linker.

3. The gene construct according to claim 1,
wherein it consists of SEQ ID NO: 1 or SEQ ID NO: 2.

4. A recombinant expression vector comprising the gene construct of claim 1.

5. An anti-cancer recombinant strain that is transformed with the gene construct according to claim 1 operably linked to an inducible promoter, wherein the strain is characterized by expressing and secreting flgM-Stv with biotin binding ability and targeting tumor cells.

6. The anti-cancer recombinant strain according to claim 5,
wherein the strain is an attenuated Salmonella strain.

7. The anti-cancer recombinant strain according to claim 6,
wherein the strain has inhibited or lost motility when expressing fusion protein of flgM and streptavidin by the inducible promoter.

8. The anti-cancer recombinant strain according to claim 7,
wherein the strain is the strain with deposit number KCTC15482BP.

9. A method of treating a cancer, comprising:
administering an anti-cancer composition comprising the anti-cancer recombinant strain of claim 5 to a subject.

10. An anti-cancer adjuvant characterized by comprising the recombinant strain of claim 5 and being co-administered with an anti-cancer agent in biotin-bound form.

11. The anti-cancer adjuvant according to claim 10,
wherein the anti-cancer agent is targeted to tumor cells and acts as a sustained-release formulation.

12. A tumor imaging adjuvant characterized by comprising the recombinant strain of claim 5 and being co-administered with a contrast agent in biotin-bound form.

13. The tumor imaging adjuvant according to claim 12,
wherein the contrast agent is one or more selected from the group consisting of radioactive nuclides, fluorescent labels, enzyme labels, chemiluminescent markers, gold preparations and magnetic preparations.

14. A method for providing information for cancer diagnosis comprising:
(A) targeting tumor cells by administering the tumor imaging adjuvant of claim 12 to a patient suspected of having cancer or undergoing anti-cancer treatment;
(B) inducing expression of flgM and streptavidin fusion protein in the recombinant strain of the tumor imaging adjuvant;
(C) administering a contrast agent in biotin-bound form; and
(D) detecting and imaging the contrast agent.


SEQUENCE LISTING (7 sequences)

SEQ ID NO: 1 - DNA, 690 bp, synthetic construct
  source 1..690: mol_type = other DNA
atgagcattgaccgtacctcacctttgaaacccgttagcactgtccagacgcgcgaaaccagcgacacgccggtacaaaaaacgcgtcaggaaaaaacgtccgccgcgacgagcgccagcgtaacgttaagcgacgcgcaagcgaagctcatgcagccaggcgtcagcgacattaatatggaacgcgtcgaagcattaaaaacggctatccgtaacggtgagttaaaaatggatacgggaaaaatagcagactcgctcattcgcgaggcgcagagctacttacagagtaaaaaacatcatcatcatcatcatagcggcggcgaatttgcgagcgcggaagcgggcattaccggcacctggtataaccagcatggcagcacctttaccgtgaccgcgggcgcggatggcaacctgaccggccagtatgaaaaccgcgcgcagggcaccggctgccagaacagcccgtataccctgaccggccgctataacggcaccaaactggaatggcgcgtggaatggaacaacagcaccgaaaactgccatagccgcaccgaatggcgcggccagtatcagggcggcgcggaagcgcgcattaatacccagtggaacctgacctatgaaggcggcagcggcccggcgaccgaacagggccaggatacctttaccaaagtgaaaccgagcgcggcgagc

SEQ ID NO: 2 - DNA, 708 bp, synthetic construct
  source 1..708: mol_type = other DNA
atgagcattgaccgtacctcacctttgaaacccgttagcactgtccagacgcgcgaaaccagcgacacgccggtacaaaaaacgcgtcaggaaaaaacgtccgccgcgacgagcgccagcgtaacgttaagcgacgcgcaagcgaagctcatgcagccaggcgtcagcgacattaatatggaacgcgtcgaagcattaaaaacggctatccgtaacggtgagttaaaaatggatacgggaaaaatagcagactcgctcattcgcgaggcgcagagctacttacagagtaaaaaaggcggcagcagcaccagccatcatcatcatcatcatagcggcggcgaatttgcgagcgcggaagcgggcattaccggcacctggtataaccagcatggcagcacctttaccgtgaccgcgggcgcggatggcaacctgaccggccagtatgaaaaccgcgcgcagggcaccggctgccagaacagcccgtataccctgaccggccgctataacggcaccaaactggaatggcgcgtggaatggaacaacagcaccgaaaactgccatagccgcaccgaatggcgcggccagtatcagggcggcgcggaagcgcgcattaatacccagtggaacctgacctatgaaggcggcagcggcccggcgaccgaacagggccaggatacctttaccaaagtgaaaccgagcgcggcgagc

SEQ ID NO: 3 - protein, 123 aa, synthetic construct
EFASAEAGITGTWYNQHGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAAS

SEQ ID NO: 4 - protein, 10 aa, synthetic construct
GSDYKDDDDK

SEQ ID NO: 5 - protein, 9 aa, synthetic construct
HHHHHHSGG

SEQ ID NO: 6 - protein, 15 aa, synthetic construct
GGSSSSHHHHHHSGG

SEQ ID NO: 7 - DNA, 14 bp, synthetic construct
  source 1..14: mol_type = other DNA
aggaggtttgacct